(19) United States
(12) Patent Application Publication
(10) Pub. No.: US 2017/0274012 A1
(43) Pub. Date: Sep. 28, 2017

(54) Acceleration of Reconstitution of Plasma Powder by Mixing with Small Beads

(71) Applicant: ENTEGRION, INC., RESEARCH TRIANGLE PARK, NC (US)

(72) Inventors: ARTHUR PALFREY BODE, RALEIGH, NC (US); OLEG GORKUN, RALEIGH, NC (US)

(73) Assignee: ENTEGRION, INC., RESEARCH TRIANGLE PARK, NC (US)

(21) Appl. No.: 15/505,798

(22) PCT Filed: Sep. 2, 2015
(86) PCT No.: PCT/US15/48071

§ 371 Date: Feb. 22, 2017

Related U.S. Application Data
(60) Provisional application No. 62/045,160, filed on Sep. 3, 2014.

(51) Int. Cl. A61K 35/16; C08L 69/00; A61J 1/12

(52) U.S. Cl. CPC A61K 35/16; A61J 1/12; C08L 69/005; B29L 2031/7734

(57) ABSTRACT

Methods of accelerating the reconstitution of plasma powder by mixing the powder with small beads is disclosed. The beads can be, for example, glass and/or plastic resin beads having a diameter from about 2 mm to about 6 mm. The plasma powder can be, for example, a spray-dried plasma powder. A method is provided of accelerating the reconstitution of a dry protein powder in a fluid that includes, but is not limited to, the steps of providing both a dry protein powder and small beads in a container, adding a reconstitution fluid to the container, and manipulating the dry protein powder and the small beads to dissolve the dry protein powder in the reconstitution fluid.

### CROSS-REFERENCE TO RELATED APPLICATIONS

The presently disclosed subject matter is related to Bode et al., U.S. Provisional Patent App. No. 62/045160, entitled “Acceleration of Reconstitution of Plasma Powder by Mixing with Small Beads,” filed on Sep. 3, 2014; the entire disclosure of which is incorporated herein by reference.

### TECHNICAL FIELD

The presently disclosed subject matter relates generally to methods of reconstituting dry protein powder and more particularly to a method of accelerating reconstitution of plasma powder (e.g., spray-dried plasma powder) by mixing the powder with small beads.

### BACKGROUND

Spray drying is a method of transforming material in a fluid state into a dried particulate form by spraying a feed of a material into a warm drying medium. Spray-dried plasma intended for transfusion is reconstituted by introduction of a liquid fluid into a closed plastic bag containing the dry powder; however, there is a tendency for the powder to clump when first wetted and to form a clay instead of dispersing easily and dissolving quickly. With vigorous manual manipulation of the bag to try to press the powder clumps into dispersion and dissolution, it can still take from about five to about eight minutes, for example, to achieve full clarity of the plasma as the particles go back into solution. This is unacceptable in situations where the plasma transfusion is being done in urgency, or when there are minimal personnel present to care for the patient and to prepare all medicines and procedures for acute care. It would be far better for the medical practitioners and the patient if the spray-dried plasma could be reconstituted more easily and more quickly. Small objects, such as spheres or other shapes, have been used to mix in with a powder in order to mill or grind down the powder into smaller pieces, such as sifted flour. This practice is common in metalworking or paint manufacture, but not common in biological applications.

Shearing particles or beads have been used in biological applications in wet cell suspensions to cause their structural disintegration or homogenization in order to harvest some internal component(s) or particles (Goldberg, 2008). In addition, porous beads are well known in the art for the separation of molecules based on size, such as in gel-filtration chromatography. Also, beads are commonly used in protein purification, such as in affinity chromatography, by covalently attaching a specific molecule to the beads that has affinity for a specific type of protein. These biological applications, however, are generally performed in liquid environments.

### SUMMARY

In one aspect, the present invention discloses a method for accelerating reconstitution of a dry protein powder in a fluid, the method comprising: a) providing a dry protein powder in a container and adding small beads to the dry protein powder in the container or providing small beads in a container and adding a dry protein powder to the small beads in the container; b) adding a reconstitution fluid to the container; and c) manipulating the dry protein powder and the small beads to dissolve the dry protein powder in the reconstitution fluid.

The dry protein powder can be a spray-dried plasma powder and there may be approximately 600 to approximately 1300 small beads for every approximately 16 grams of spray-dried plasma powder.

Manipulating the dry protein powder and the small beads can be performed by manipulating the powder and the beads from the outside of the container. The container can be a plastic bag and the plastic bag can be a plasma bag.

The small beads can comprise at least one material selected from the group consisting of glass and plastic resin and the small beads can comprise at least one material selected from the group consisting of polycarbonate, polypropylene, polyvinyl chloride, cellulose acetate, and borosilicate glass. The small beads can be approximately spherical or approximately oval. The small beads can have diameters from about 2 mm to about 6 mm, more particularly the small beads can have diameters from about 2 mm to about 4 mm, and more particularly the small beads can have diameters from about 4 mm to about 5 mm. The small beads can be pre-sterilized.

In the method, adding the reconstitution fluid to the container can wet substantially all of the dry protein powder.

In another aspect, the present invention discloses a composition comprising small beads with a diameter from about 2 mm to about 6 mm and a dry protein powder. The dry protein powder can be spray-dried plasma powder.

The composition can be in a plasma bag. The small beads in the composition can comprise at least one material selected from the group consisting of glass and plastic resin and the small beads can comprise at least one material selected from the group consisting of polycarbonate, polypropylene, polyvinyl chloride, cellulose acetate, and borosilicate glass.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1, FIG. 2 and FIG. 3 illustrate plan views of an example of a container used for the reconstitution of a dry protein powder and a process of accelerating the reconstitution of the dry protein powder in a fluid;

FIG. 4 illustrates a flow diagram of an example of a method of accelerating the reconstitution of a dry protein powder in a fluid;

FIG. 5 shows a plot of thrombin generation of Ked05 samples assayed immediately after reconstitution in citric acid/PO₄ buffer; and

FIG. 6 shows a plot of Factor V levels from reconstitution samples using different spray-dried powder and reconstitution in WFI buffer.

### DETAILED DESCRIPTION

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The presently disclosed subject matter provides compositions for and methods of using small beads in combination with a dry protein powder suspension to aid in reconstitution of the powder. In some embodiments, the present invention provides methods of accelerating the reconstitution of plasma powder (e.g., spray-dried plasma powder) by mixing the powder with small beads. Namely, in some embodiments, the present invention provides methods to facilitate the mixing of spray-dried plasma with reconstitution fluid by the addition of small beads. The reconstituted plasma may be used for transfusion into a subject.

Small objects have been used to mix in with a dry powder in order to mill or grind down the powder into smaller pieces. In biological applications, small objects, such as small beads, have been used for homogenizing wet cell suspensions in order to separate a specific cell component from the rest of the cell and for separation of specific molecules from other molecules in a liquid sample. However, small objects have not been used for aiding in the dissolution of powders during the preparation of solutions as disclosed herein. The present invention provides methods of using beads in combination with a dry protein powder, such as spray-dried plasma powder, to break down the wetted clumps after fluid is added.

By “accelerating reconstitution,” it is meant that the dry protein powder is dissolved in the fluid faster than if small beads were not added to the container. Namely, “accelerating reconstitution” means that the reconstitution time is reduced when the small beads are added to the container as compared with the reconstitution time in the absence of the small beads. Reconstitution completion can be seen by clarity of the solution after the protein powder has dissolved in the fluid. The time for reconstitution completion may decrease in the presence of small beads by more than 10%, 20%, 30%, 40%, 50%, 60%, 70%, 80%, 90%, or even close to 100% as compared to the time for reconstitution completion in the absence of small beads. In some embodiments, the reconstitution time decreases by approximately 70%.

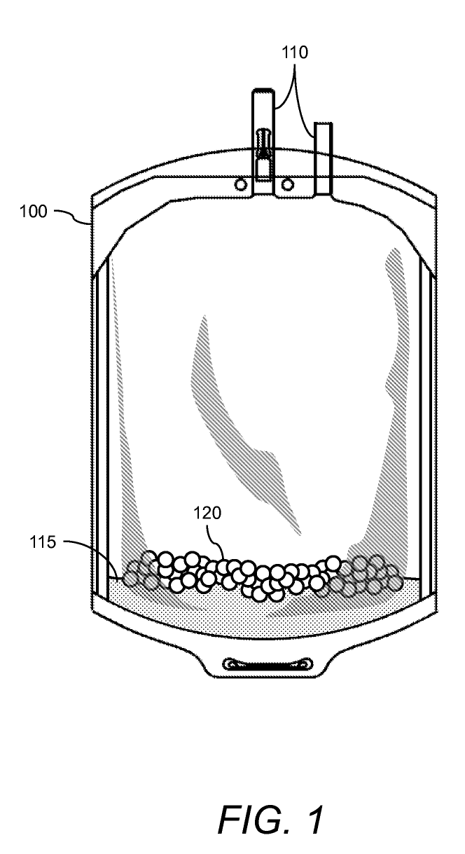

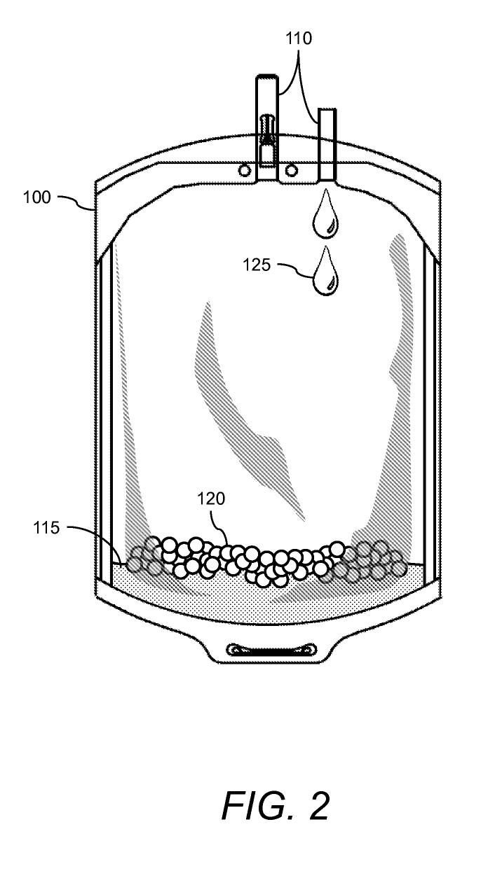

FIG. 1, FIG. 2 and FIG. 3 illustrate plan views of an example of a container used for the reconstitution of a dry protein powder and a process of accelerating the reconstitution of the dry protein powder in a fluid. In this example, FIG. 1, FIG. 2 and FIG. 3 show a container 100 in which the dry protein powder can be reconstituted in a fluid. As used herein, the term “container,” refers to a three-dimensional object, i.e., a receptacle, having a hollow interior or an interior capable of containing substances. In some embodiments, the container is a plastic bag. In other embodiments, the plastic bag is a plasma bag, a transfusion bag, a platelet storage bag, an intravenous (IV) bag, a blood transfer bag, and the like. The term “plasma bag” as used herein refers to a plastic bag that can be used for storing or holding plasma. In some embodiments, the plasma bag comprises connections for IV tubes.

In the example shown in FIG. 1, FIG. 2 and FIG. 3, the container 100 is a plastic bag, such as a plasma bag, a transfusion bag, a platelet storage bag, an IV bag, a blood transfer bag, and the like. Further, the container 100 has one or more fluid ports 110 (e.g., connections for IV tubes) through which any fluid can be added to or removed from the container 100. Referring now to FIG. 1, a certain amount of dry protein powder 115 and a certain number or volume of beads 120 are provided inside the container 100.

The dry protein powder 115 used in the methods of the present invention can be any dry protein powder that is capable of being reconstituted. In some embodiments, the dry protein powder 115 comprises blood products, such as whole blood, blood plasma, blood platelets, red blood cells, blood serum, and the like, as well as combinations of these. In a preferred embodiment, the dry protein powder 115 is spray-dried plasma powder.

In some embodiments, the beads 120 used in the methods of the present invention comprise at least one material selected from the group consisting of glass and/or plastic resin. In other embodiments, the beads 120 comprise an inert plastic resin, such as polycarbonate, polypropylene, polyvinyl chloride, cellulose acetate, borosilicate glass, and the like. In a preferred embodiment, the beads 120 comprise polycarbonate. In still other embodiments, the beads 120 comprise at least one material selected from the group consisting of polycarbonate, polypropylene, polyvinyl chloride, cellulose acetate, and borosilicate glass. In further embodiments, the beads 120 are approximately spherical or approximately oval. In a preferred embodiment, the beads 120 are approximately spherical. In still further embodiments, the beads 120 can have diameters from about 2 mm to about 6 mm, such as from about 2 mm to about 4 mm or from about 4 mm to about 5 mm.

Preferred characteristics of the beads 120 used in the present invention include, but are not limited to, high tensile strength so as to prevent rupture or fragmentation during handling, a non-toxic profile in terms of leachable substances, and beads 120 that are easily sterilized. In some embodiments, the beads 120 are pre-sterilized.

Referring now to FIG. 2, a volume of reconstitution fluid 125 is added (via a port 110) to the dry protein powder 115 and the beads 120 inside container 100. The components of the reconstitution fluid 125 depends on the protein(s) in the dry protein powder, the pH required for the use of the reconstituted protein(s), the function of the reconstituted protein(s) in solution, and the like. In some embodiments, the reconstitution fluid 125 is sterile so that the reconstituted protein may also be in a sterile environment. In other embodiments, the reconstitution fluid 125 is a buffered solution. In still other embodiments, the reconstitution fluid 125 comprises distilled water, saline solution, and/or glycine.

In some embodiments, adding the reconstitution fluid 125 to the container wets substantially all of the dry protein powder 115. In other embodiments, manipulating the dry protein powder 115 and the beads 120 is performed by manipulating (e.g., by hand) the dry protein powder 115 and the beads 120 from the outside of the container 100. When reconstitution fluid 125 is added into the container 100, the beads 120 act as a milling surface to help contact the dry protein powder 115 with the fluid in spread fashion and prevent the formation of clay or clumps which are harder to wet thoroughly and disperse. As the container is manipulated, the beads 120 mix with the dry protein powder 115 and reconstitution fluid 125 and press against each other and the internal container walls to mill and grind the dry protein powder 115 into smaller pieces that are more easily wetted and thus more quickly dissolved.

When the dry protein powder 115 is fully reconstituted, a volume of reconstituted plasma solution 130 is present in the container 100, as shown in FIG. 3. In some embodiments, the dry protein powder 115 is physiologically active plasma powder and reconstituting it forms physiologically active reconstituted plasma.

In some embodiments, the beads 120 are mixed in with the dry protein powder 115 produced by spray-drying of liquid plasma at the time of sterile fill into the final container, such as a plastic bag, followed by sealing. The selected beads 120 are inert to interactions with the coagulation or complement activation systems in plasma and thus do not affect its stability as a pharmaceutical transfusion product.

In some embodiments, the methods of the present invention comprise adding a certain number of pre-sterilized beads 120 by weight into the container 100 as it is being filled with the dry protein powder 115, the container 100 then being sealed with both the powder and bead components contained within, and entered into distribution and storage as a single use unit. In these embodiments, the beads 120 would be considered part of the packaging of the spray-dried plasma powder but not an element of the drug substance itself.

In some embodiments, the preferred embodiment of the present invention would be the use of a totally inert plastic resin, such as polycarbonate, with optimal “feel” beads, such as beads 120 having a diameter of from about 4 mm to about 5 mm, in minimal effective ratio with the spray-dried powder with the optional property of floatation in the reconstituted plasma solution. In other embodiments, there is about 500 to about 5000 beads 120 for every from about 2 grams to about 50 grams of dry protein powder 115. In one example, there is about 600 to about 1,300 beads 120 for every approximately 16 grams of spray-dried plasma powder.

The present invention also provides compositions comprising the beads 120 and dry protein powder 115. In some embodiments, a composition comprising the beads 120 with a diameter from about 2 mm to about 6 mm and a dry protein powder 115 is provided. In other embodiments, the dry protein powder 115 is spray-dried plasma powder. In still other embodiments, the composition is in a plasma bag. In further embodiments, the beads 120 comprise at least one material selected from the group consisting of glass and plastic resin.

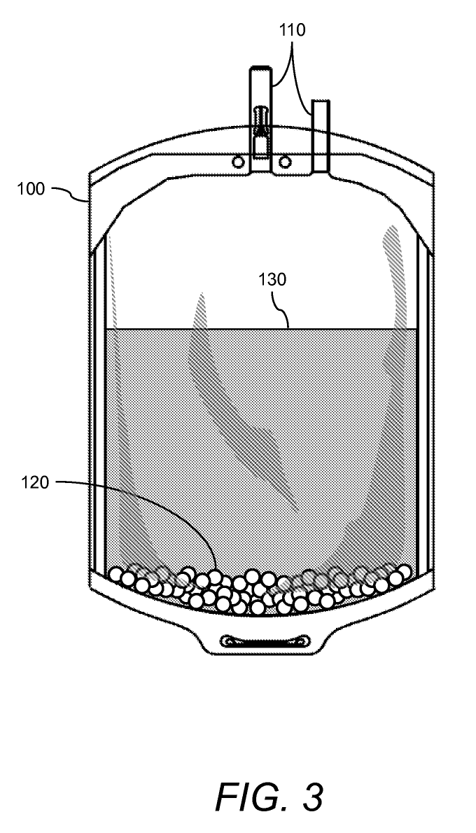

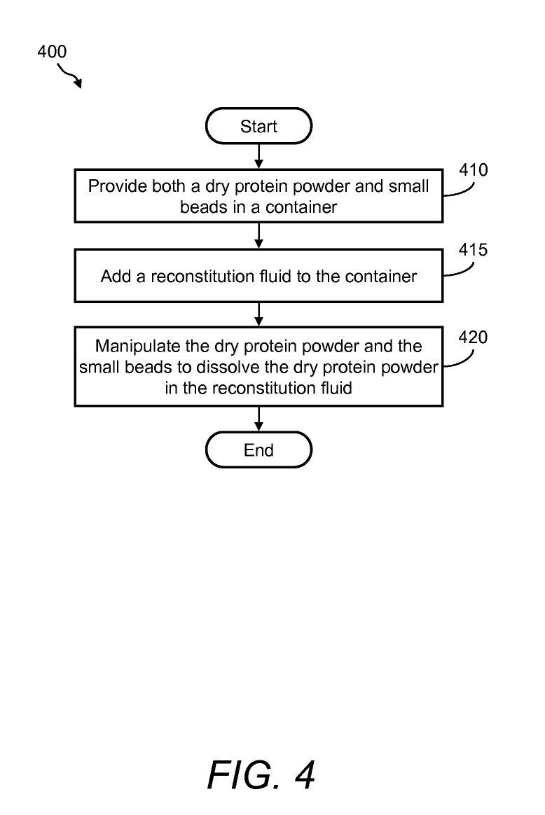

FIG. 4 illustrates a flow diagram of an example of a method 400 of accelerating the reconstitution of a dry protein powder (e.g., the dry protein powder 115) in a fluid (e.g., the reconstitution fluid 125). The method 400 may include, but is not limited to, the following steps.

At a step 410, both a dry protein powder and small beads are provided in a container. For example and referring now to FIG. 1, both the dry protein powder 115 and the beads 120 are provided in the container 110. In one example, the dry protein powder 115 is added to the container 110 and then the beads 120 are added to the container 110. In another example, the beads 120 are added to the container 110 and then the dry protein powder 115 is added to the container 110. In another example, the dry protein powder 115 and the beads 120 are mixed outside of the container 100 and then added simultaneously to the container 110. In the case of the container 100 being a plastic bag, once the dry protein powder 115 and the beads 120 are in the plastic bag, the plastic bag is sealed.

At a step 415, a reconstitution fluid is added to the container. For example and referring now to FIG. 2, the reconstitution fluid 125 is added to the container 110 (e.g., the plastic bag) via a port 110.

At a step 420, the dry protein powder and the small beads are manipulated to dissolve the dry protein powder in the reconstitution fluid. For example and referring now to FIG. 2, the dry protein powder 115 and the beads 120 are manipulated to dissolve the dry protein powder 115 in the reconstitution fluid 125. The manipulation can be, for example, by squeezing, rubbing, and/or working by hand the dry protein powder 115 and the beads 120 inside the container 100. In so doing, a milling action occurs between the dry protein powder 115, the beads 120, and the container 100. FIG. 3 shows an example of the dry protein powder 115 when fully reconstituted to form the reconstituted plasma solution 130 inside the container 100.

Using the method 400, the dry protein powder 115 can be reconstituted in about 2 minutes or less, which is a much shorter reconstitution time as compared with the about 5 to about 8 minutes that conventional reconstitution methods (absent small beads) require.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### EXAMPLE 1

### Reconstitution Studies

Resusix® (available from Entegrion, Inc., Research Triangle Park, N.C.), a spray-dried plasma powder, was used in the reconstitution studies. Resusix® is composed of small protein particles of higher density than a lyophilization cake. Initial studies showed that the reconstitution fluid should be introduced in a highly dispersive manner to avoid clay-formation and promote wetting of all powder. It was also shown that the form factor of the powder bag and the volume ratio with the reconstitution fluid were important practical details: low fluid-fill versus bag volume allowed the bag sides to be pressed together to help “knead” the powder into solution. It was also important to remove air from the bag before the buffer fill for the same reason. Solid beads were introduced into the powder bag before the reconstitution fluid to promote “milling” of the powder during reconstitution with less strenuous manual manipulation.

Materials tested to date include 1.0-mm borosilicate glass beads, 2.0-mm cellulose acetate (CA) beads, 3.0-mm cellulose acetate (CA) beads, 3.18-mm polypropylene (PP) beads, and 4.76-mm polyvinylchloride (PVC) beads.

16 gm Kedy001 Resusix® in an ethylene vinyl acetate (EVA) bag or 2.4 gm Ked05SDP (spray-dried in house) in a PediPak bag were reconstituted in citric/PO₄ buffer or WFI buffer in the presence or absence of beads. The samples were tested immediately or after three hours at room temperature (RT) to examine effects on plasma protein activation or degradation. Testing included the reconstitution time, a coagulation panel, the amount of C3a desArg and C5a desArg proteins, and thrombin generation using Thrombinoscope software.

Table 1 below shows results from an experiment using 50 g and 100 g of borosilicate glass beads with 16 g Kedy001 Resusix® reconstituted in WFI buffer. Namely, Table 1 shows data from an embodiment of the methods of the present invention in which no beads (Sample 1), 100 g of 1-mm borosilicate glass beads (Sample 2), or 50 g of 1-mm borosilicate glass beads (Sample 3) were added to Kedy001 Resusix® powder for reconstitution in WFI buffer.

Glass beads greatly sped up reconstitution of the spray-dried plasma. Although the Kedy001 spray-dried plasma was low in the levels of Factor XI, glass beads further accelerated the loss. The lower levels of Factor XI resulted in an increase in the activated partial thromboplastin time (aPTT). In addition, the borosilicate glass beads did not appear to cause an increase in complement activation as measured by the C3a/C5a desArg proteins. Other experiments in smaller bags with better form factors and citric acid/PO₄ buffer confirmed that the glass beads accelerated reconstitution of the Kedy001 Resusix® powder, but the low initial Factor XI levels made this particular powder unsuitable for continued analysis.

TABLE 1
Test results of Glass beads in WFI buffer

| Sample | CONTROL | | Sample 2 | | Sample 3 | |
|---|---|---|---|---|---|---|
| Description | Kedy001 16 g | | Kedy001 | | Kedy001 | |
| | | | 16 g + 100 g | | 16 g + 50 g | |
| | | | Borosilicate | | Borosilicate | |
| | | | beads (1 mm) | | beads (1 mm) | |
| Concentration | 1X | | 1X | | 1X | |
| Buffer | WFI | | WFI | | WFI | |
| Time to Clarify | 14 min | | 3 min 11 sec | | 5 min 30 sec | |
| Sampling Time | 30 min | 3 hr | 30 min | 3 hr | 30 min | 3 hr |
| pH | 8.87 | 8.93 | 8.83 | 8.89 | 8.88 | 8.92 |
| PT (sec) | 15.7 | 16.5 | 15.2 | 16 | 15.4 | 16 |
| aPTT (sec) | 42.3 | 45.2 | 47.3 | 55.5 | 45.2 | 51.6 |
| Fibrinogen | 244 | 252 | 238 | 254 | 233 | 249 |
| (mg/dl) | | | | | | |
| Factor II (%) | 73 | 75 | 74 | 70 | 71 | 77 |
| Factor V (%) | 59 | 44 | 61 | 45 | 60 | 44 |
| Factor VII (%) | 120 | 119 | 122 | 139 | 114 | 121 |
| Factor VIII (%) | 58 | 56 | 55 | 59 | 54 | so |
| Factor IX (%) | 88 | 91 | 81 | 96 | 80 | 98 |
| Factor X (%) | 85 | 74 | 87 | 77 | 87 | 77 |
| Factor XI (%) | 19 | 22 | 11 | 5 | 13 | 11 |
| Factor XII (%) | 82 | 93 | 79 | 91 | 79 | 84′ |
| Protein C (%) | 117 | 117 | 115 | 120 | 114 | 117 |
| Protein S Activity | 45 | 41 | 40 | 39 | 42 | 42 |
| (%) | | | | | | |
| Free Protein S (%) | 68 | 67 | 70 | 67 | 73 | 65 |
| AT III (%) | 90 | 93 | 90 | 89 | 84 | 90 |
| AntiPlasmin (%) | 23 | 26 | 22 | 24 | 21 | 23 |
| Fibrin Monomer | 6.91 | 2.92 | 5.56 | 4.82 | 5.39 | 2.05 |
| (ug/mL) | | | | | | |
| VWF: Ag (%) | 153 | 164 | 156 | 165 | 161 | 170 |
| Total Protein | 57.7 | 56.7 | 55.5 | 55 | 56.6 | 55.8 |
| (mg/mL) | | | | | | |
| C3a desArg | 31,000 | 26.000 | 23,000 | 29,000 | 30,000 | 30,000 |
| (ng/mL) | | | | | | |
| C5a desArg | 174 | 171 | 154 | 147 | 142 | 145 |
| (ng/mL) | | | | | | |

The next experiment was performed using Ked05 in-house spray-dried powder and better form factor of the bag. Namely, a comparison experiment of glass beads with cellulose acetate beads using the methods of the present invention since cellulose acetate beads are more blood-compatible. Results from this experiment showed that both the glass beads and the cellulose acetate beads accelerated reconstitution of the spray-dried plasma. However, the glass beads still accelerated the loss and/or instability of Factor XI but the cellulose acetate beads did not, as shown in Table 2 below. Namely, Table 2 shows data from an embodiment of the methods of the present invention in which no beads (Sample 1), 2-mm cellulose acetate beads (Sample 2), or 1-mm borosilicate glass beads (Sample 3) were added to Ked05 in-house spray-dried powder for reconstitution in citric acid/PO₄ buffer. The lower levels of Factor XI with the glass beads caused an increase in the activated partial thromboplastin time (aPTT). In addition, results suggested that the cellulose acetate beads did not cause an increase in the C3a desArg protein levels.

TABLE 2
Comparison of Glass beads to Cellulose Acetate and Borosilicate beads

| Sample | CONTROL | | Sample 2 | | Sample 3 | |
|---|---|---|---|---|---|---|
| Description | 1.6 g | | 1.6 g | | 1.6 g | |
| | Ked05SDP | | Ked05SDP + | | Ked05SDP + | |
| | | | Cellulose | | Borosilicate | |
| | | | Acetate | | beads | |
| | | | beads (2 mm) | | (1 mm) | |
| Concentration | 1X | | 1X | | 1X | |
| Buffer | 20 mL Citric | | 20 mL Citric | | 20 mL Citric | |
| | Acid/Phos | | Acid/Phos | | Acid/Phos | |
| | buffer | | buffer | | buffer | |
| | pH 3.2 | | pH 3.2 | | pH 3.2 | |
| Time to Clarify | 1 min 45 sec | | 1 min 5 sec | | 1 min 12 sec | |
| Sampling Time | 4 min | 3 hr | 4 min | 3 hr | 4 min | 3 hr |
| PT (sec) | 16.4 | 15.2 | 16.3 | 15.8 | 15.9 | 15.3 |
| aPTT (sec) | 36 | 35.2 | 36.6 | 36.6 | 36.7 | 40.8 |
| Fibrinogen | 285 | 290 | 277 | 282 | 280 | 301 |
| (mg/dl) | | | | | | |
| Factor II (%) | 77 | 82 | 77 | 79 | 76 | 85 |
| Factor V (%) | 79 | 91 | 82 | 84 | 88 | 93 |
| Factor VII (%) | 82 | 81 | 77 | 88 | 78 | 90 |
| Factor VIII (%) | 60 | 51 | 55 | 55 | 58 | 59 |
| Factor IX (%) | 93 | 92 | 88 | 85 | 76 | 86 |
| Factor X (%) | 83 | 83 | 83 | 83 | 84 | 89 |
| Factor XI (%) | 44 | 45 | 38 | 36 | 42 | 20 |
| Factor XII (%) | 81 | 73 | 76 | 71 | 74 | 73 |
| Protein C (%) | 107 | 108 | 106 | 106 | 109 | 109 |
| Free Protein S (%) | 67 | 67 | 67 | 63 | 67 | 66 |
| AT III (%) | 97 | 95 | 95 | 92 | 97 | 96 |
| AntiPlasmin (%) | 47 | 46 | 43 | 44 | 46 | 53 |
| Fibrin Monomer | 6.35 | 5.72 | 4.79 | 9.38 | 3.73 | 6.27 |
| (ug/mL) | | | | | | |
| VWF: Ag (%) | 142 | 143 | 143 | 141 | 141 | 141 |
| Total Protein | 53.5 | 52.9 | 53 | 52.9 | 54.4 | 54.9 |
| (mg/mL) | | | | | | |
| C3a desArg | 16,000 | 18,000 | 17,000 | 20,000 | 19,000 | 20,000 |
| (ng/mL) | | | | | | |

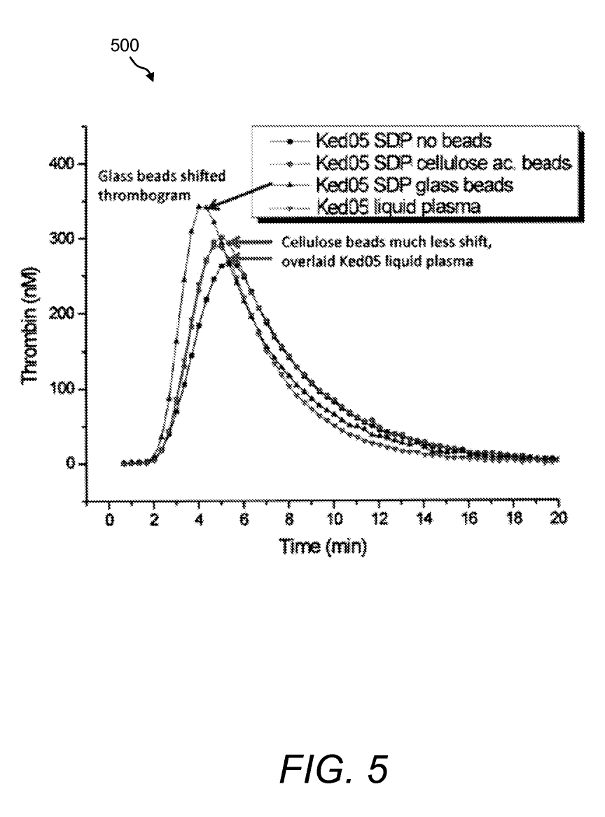

Thrombin generation experiments immediately after reconstitution of Ked05 spray-dried plasma (SDP) in citric acid/PO₄ buffer with glass and cellulose acetate beads showed that the glass beads shifted the thrombogram whereas the cellulose acetate beads showed much less of a shift as compared to the liquid plasma. Referring now to FIG. 5 is a plot 500 of thrombin generation of Ked05 samples assayed immediately after reconstitution in citric acid/PO₄ buffer. Similar results were obtained with reconstitution in WFI buffer. Because the glass beads seemed too active, cellulose or other plastic materials were used for further experiments.

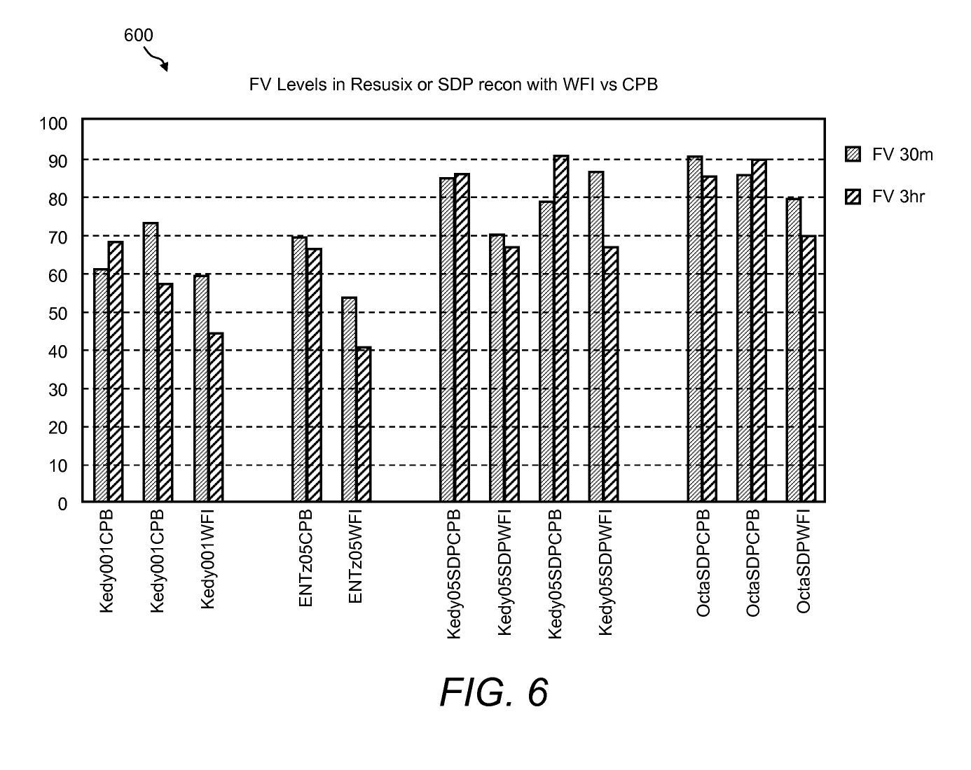

The effectiveness of WFI buffer and citric acid/PO₄ buffer (CPB) were compared to determine which buffer was more suitable for reconstitution of the spray-dried plasma powder. Referring now to FIG. 6 is a plot 600 of Factor V levels from reconstitution samples using different spray-dried powder and reconstitution in WFI buffer. After reconstitution of several spray-dry plasma powders (from Nova labs or in-house), a trend was seen towards lower recoveries or instability of Factor V (FV) in the WFI buffer (pH at reconstitution=8.7 to 8.9). The other factors were stable in the WFI buffer. Therefore, further experiments were mainly performed using the citric acid/PO₄ buffer.

A comparison of cellulose beads to polypropylene beads and to PVC beads can be seen in Table 3 below. Namely, Table 3 shows data from an embodiment of the methods of the present invention in which no beads (Sample 1), 3-mm cellulose acetate beads (Sample 2), 3.18-mm polypropylene beads (Sample 3), and 4.76-mm PVC beads were added to Ked05SDP spray-dried powder for reconstitution in citric acid/PO₄ buffer (CPB).

Materials for this experiment were selected for availability and not matched for material and size of the beads. The samples with beads showed an acceleration of reconstitution time although it appeared to be variable. Factor XI seemed to be unstable in the control sample and the samples with beads. The results were unusual due to the instability of Factor XI even in the control sample, but results suggested that a bead size from about 2 mm to about 4 mm was optimum for reconstitution of spray-dried plasma in this bag form factor.

Surprisingly, none of the beads in the experiments appeared to cause an increase in complement activation as measured by C3a/C5a desArg protein levels, nor was there an activation of Factor VII or Factor XII.

TABLE 3
Comparison of Cellulose Acetate beads to Polypropylene and PVC beads

| Sample | CONTROL | | Sample 2 | | Sample 3 | | Sample 4 | |
|---|---|---|---|---|---|---|---|---|
| Description | 2.4 g | | 2.4 g | | 2.4 g | | 2.4 g | |
| | Ked05SDP | | Ked05SDP + | | Ked05SDP + | | Ked05SDP + | |
| | | | Cellulose | | Polypropylene | | PVC beads | |
| | | | Acetate beads | | beads | | (4.76 mm) | |
| | | | (3 mm) | | (3.18 mm) | | | |
| Concentration | 1X | | 1X | | 1X | | 1X | |
| Buffer | 30 mL CPB | | 30 mL CPB | | 30 mL CPB | | 30 mL CPB | |
| | pH 3.4 | | pH 3.4 | | pH 3.4 | | pH 3.4 | |
| Time to | 1 min 50 sec | | 45 sec | | 1 min 20 sec | | 1 min 30 sec | |
| Clarify | | | | | | | | |
| Sampling | 4 min | 3 hr | 4 min | 3 hr | 4 min | 3 hr | 4 min | 3 hr |
| Time | | | | | | | | |
| pH | 7.06 | 7.27 | 7.02 | 7.25 | 7.06 | 7.26 | 7.06 | 7.26 |
| PT (sec) | 16.4 | 15.7 | 16.9 | 15.9 | 16.7 | 16 | 16.5 | 15.8 |
| aPTT (sec) | 39 | 38.1 | 40.4 | 42.6 | 39.3 | 38.8 | 39.5 | 39.4 |
| Fibrinogen | 279 | 287 | 260 | 258 | 281 | 275 | 276 | 27 |
| (mg/dl) | | | | | | | | |
| Factor II (%) | 84 | 82 | 83 | 79 | 81 | 83 | 79 | 77 |
| Factor V (%) | 84 | 93 | 86 | 92 | 92 | 85 | 84 | 83 |
| Factor VII | 76 | 88 | 82 | 86 | 84 | 84 | 83 | 86 |
| (%) | | | | | | | | |
| Factor VIII | 71 | 61 | 65 | 56 | 67 | 64 | 68 | 64 |
| (%) | | | | | | | | |
| Factor IX (%) | 93 | 95 | 81 | 85 | 85 | 88 | 88 | 88 |
| Factor X (%) | 84 | 91 | 74 | 83 | 83 | 81 | 79 | 78 |
| Factor XI (%) | 54 | 46 | 46 | 30 | 49 | 40 | 48 | 35 |
| Factor XII | 73 | 72 | 69 | 68 | 78 | 74 | 75 | 69 |
| (%) | | | | | | | | |
| Protein C (%) | 101 | 101 | 97 | 100 | 101 | 104 | 101 | 101 |
| Free Protein S | 68 | 62 | 62 | 63 | 64 | 63 | 61 | 62 |
| (%) | | | | | | | | |
| AntiPlasmin | 46 | 48 | 44 | 47 | 50 | 52 | 48 | 47 |
| (%) | | | | | | | | |
| Total Protein | 53.4 | 52 | 52 | 51.1 | 54.4 | 54.8 | 52.8 | 54.3 |
| (mg/mL) | | | | | | | | |

### Example 2

In this example, inert particles were added to the spray-dried plasma powder, wherein the inert particles will at least increase the surface area over which one could press or grind the powder against the inner bag walls. This maneuver was first attempted with 1-mm glass beads but it was found that the rolling of the beads together amongst the wetted powder provided a grinding action beyond just increased surface area for contact with the bag walls. This was further facilitated by the natural sticking of the powder to the glass beads, an unexpected favorable property. The total effect of the beads was to decrease the reconstitution time of the spray-dried plasma powder to two minutes or less, approximately a 70% reduction in time, as seen by the clarity of the solution. Further experimentation led to a change from glass beads to plastic resins to decrease the interactions of plasma proteins with the glass beads.

Furthermore, it was found that an increase in the size of the beads to from about 4 mm to about 5 mm allowed for introduction of fewer beads into the powder to achieve the reconstitution acceleration and gave a better “feel” for the milling effect (subjective opinion of investigators). The effort required to achieve full reconstitution decreased appreciably along with the time required to reach clarity of the solution. Some of the beads used in early experimentation were of a low enough density to actually float in the reconstituted plasma, which had the beneficial effect of separating the foam from the plasma surface, and reduced the chance of the beads entering the infusion line when docked with the plasma bag for delivery.

The polycarbonate beads were unique in the extent to which the spray-dried powder clung to the beads during the wetting and reconstitution process, and gave marginally the shortest reconstitution times.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for accelerating reconstitution of a dry protein powder in a fluid, the method comprising:
a) providing a dry protein powder in a container and adding small beads to the dry protein powder in the container or providing small beads in a container and adding a dry protein powder to the small beads in the container;
b) adding a reconstitution fluid to the container; and
c) manipulating the dry protein powder and the small beads to dissolve the dry protein powder in the reconstitution fluid.

2. The method of claim 1, wherein the dry protein powder is spray-dried plasma powder.

3. The method of claim 2, wherein there are approximately 600 to approximately 1300 small beads for every approximately 16 grams of spray-dried plasma powder.

4. The method of claim 1, wherein manipulating the dry protein powder and the small beads is performed by manipulating the powder and the beads from the outside of the container.

5. The method of claim 1, wherein the container is a plastic bag.

6. The method of claim 5, wherein the plastic bag is a plasma bag.

7. The method of claim 1, wherein the small beads comprise at least one material selected from the group consisting of glass and plastic resin.

8. The method of claim 7, wherein the small beads comprise at least one material selected from the group consisting of polycarbonate, polypropylene, polyvinyl chloride, cellulose acetate, and borosilicate glass.

9. The method of claim 1, wherein the small beads are approximately spherical or approximately oval.

10. The method of claim 1, wherein the small beads have diameters from about 2 mm to about 6 mm.

11. The method of claim 10, wherein the small beads have diameters from about 2 mm to about 4 mm.

12. The method of claim 10, wherein the small beads have diameters from about 4 mm to about 5 mm.

13. The method of claim 1, wherein the small beads are pre-sterilized.

14. The method of claim 1, wherein adding the reconstitution fluid to the container wets substantially all of the dry protein powder.

15. A composition comprising small beads with a diameter from about 2 mm to about 6 mm and a dry protein powder.

16. The composition of claim 15, wherein the dry protein powder is spray-dried plasma powder.

17. The composition of claim 15, wherein the composition is in a plasma bag.

18. The composition of claim 15, wherein the small beads comprise at least one material selected from the group consisting of glass and plastic resin.

19. The composition of claim 18, wherein the small beads comprise at least one material selected from the group consisting of polycarbonate, polypropylene, polyvinyl chloride, cellulose acetate, and borosilicate glass.
